(19) United States
(12) Patent Application Publication
(10) Pub. No.: US 2005/0181024 A1
(43) Pub. Date: Aug. 18, 2005

(54) Antimicrobial ballistic fabrics and protective articles

(76) Inventor: Subramaniam Sabesan, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/883,105

(22) Filed: Jul. 1, 2004

Related U.S. Application Data
(60) Provisional application No. 60/490,050, filed on Jul. 25, 2003.

(51) Int. Cl. A61K 9/70

(52) U.S. Cl. 424/443; 514/55; 442/123

(57) ABSTRACT

The present invention relates to methods for producing antimicrobial ballistic fabrics and protective apparel comprising aramid, polybenzazole or high-performance polyethylene, which articles are rendered antimicrobial by a chitosan agent, thereby preventing the development of odor and fungal and bacterial growth.

### FIELD OF THE INVENTION

The present invention relates to methods for producing antimicrobial ballistic fabrics and protective apparel. Such articles are rendered antimicrobial by a chitosan agent, thereby preventing the development of odor and preventing fungal and bacterial growth.

### TECHNICAL BACKGROUND OF THE INVENTION

At present, garments made of ballistic fabrics are washed by hand with cold water and mild detergent, rinsing thoroughly to remove all traces of detergent. Proper rinsing prohibits the accumulation of residual soap film, which can absorb water and reduce the ballistic resistance of certain types of ballistic fabric. While such a process is time consuming and laborious, it is necessary since machine washing or drying, either in the home or commercially can be damaging to the fabric, ultimately affecting its ballistic performance. Furthermore, some detergents, dry cleaning solvents, bleach and starch may reduce the garment's level of ballistic resistance and most manufacturers strongly recommend against their use. Furthermore, most manufacturers of ballistic fabric strongly recommend that ballistic fabrics never be submerged in water or dried outdoors, even in the shade, because ultraviolet light causes degradation of certain types of ballistic fabric.

Similarly, the care and cleaning of protective apparel, such as fire fighter turnout gear and athletic protective items such as knee pads, shin guards, shoulder pads and the like, is laborious and time consuming. Repeated cleaning can reduce the useful life of such garments.

A problem common to both protective gear and garments made of ballistic fabric is sweat from the wearer. If the sweat is not removed, fungal and/or bacterial growth can result over time. This fungal and/or bacterial growth ultimately degrades the protective properties of the article, results in noxious odors, and possibly even results in long-term health issues for the wearer. With regard to fire fighter turnout gear in particular, all fabrics and components used in the construction of fire fighter protective clothing must pass minimum performance requirements. Thus, the inner lining of protective garments designed for fire fighters and garments designed for others who work in environments where there is a danger of exposure to flame and high temperature are usually made from aramid fibers and yarns.

U.S. Patent Application Publication 2002/0115581 discloses a composition and methods for odor and fungal control in ballistic fabrics and other protective garments. The composition is essentially free of any material that would soil or stain fabric or react negatively with the ballistic fabric or other protective apparel. The composition preferably comprises cyclodextrin (as the odor-trapping agent) and a surfactant. The composition is preferably applied as small particle size droplets, especially from spray containers, directly onto fabric or clothing. Durability of such a coating is not demonstrated.

U.S. Pat. No. 5,753,008 and European Patent Application 1,038,571 each disclose polybenzamidazole membranes and hollow fibers that are rendered solvent resistant by application of a permselective, crosslinked, polymer coating such as crosslinked chitosan. The coating polymer must be crosslinked. Neither molecular weight nor degree of deacetylation of a chitosan coating are considered.

U.S. Pat. Nos. 6,139,688, 5,998,026, and 5,827,610 disclose aramid fibers 0.15 to 10 millimeters in length, with a surface area of 0.5 to 20 square meters per gram coated with chitosan. Pulp or floc comprising such fibers is used to make friction papers that have improved high temperature performance versus untreated aramid. The coated aramid fiber can be made by dispersing uncoated fiber in an aqueous solution of chitosan and adjusting the pH of the solution to precipitate the chitosan onto the fibers. Coated pulp can also be made by refining uncoated floc in a solution of chitosan to yield a dispersion of pulp in the solution; and, then, adjusting the pH to precipitate the chitosan. Coated floc can be made by co-precipitating chitosan onto a dispersion of floc. Such fibers are too short to be used in apparel applications.

Consequently, there remains a need for a reliable means of retarding or eliminating fungal and/or bacterial growth on difficult-to-wash ballistic fabrics and other protective apparel.

### SUMMARY OF THE INVENTION

The present invention relates to ballistic fabric articles and protective gear comprising aramid, polybenzazole, or high performance polyethylene fiber that is rendered antimicrobial by a chitosan agent, thereby preventing the development of odor and fungal and bacterial growth. The chitosan agent can be applied to the article directly or to the fiber or as a fabric finish easily and in a cost-effective way.

The present invention concerns a method for rendering an article comprising aramid or polybenzazole antimicrobial, comprising the sequential steps of:
(a) providing an article comprising aramid, polybenzazole;
(b) contacting said article with water;
(c) optionally, contacting the article with a basic solution;
(d) contacting the article produced in step (b) or step (c) with a solution comprising a chitosan agent selected from the group consisting of chitosan, chitosan salts and chitosan derivatives;
(e) drying the article; and
(f) optionally, heating the dried article at a temperature in the range of 30° C. to 200° C. for 30 minutes to 18 hours.

Further disclosed is a continuous method for rendering an article comprising aramid or polybenzazole antimicrobial, comprising the sequential steps of:
(a) providing a feed station on which is disposed the article and a take-up station capable of receiving the article;
(b) drawing the article from the feed station through a first treatment station wherein said article is wet with water;
(c) optionally, drawing the article from the first treatment station through a second treatment station wherein said article is exposed to a basic solution;
(d) drawing the step (b)- or step (c)-treated article through a third treatment station wherein the article is exposed to a solution comprising a chitosan agent selected from the group consisting of chitosan, chitosan salts and chitosan derivatives;
(e) drawing the step (c)- or step (d)-treated article through a drying station;
(f) optionally, heating the step (e)-treated article at a temperature in the range of 30° C. to 200° C. for 30 minutes to 18 hours, after it exits the drying station; and
(g) causing the step (e)- or step (f)-treated article to be received on and accumulate on the take-up station.

Further disclosed are antimicrobial chitosan-treated ballistic and protective articles.

### BRIEF DESCRIPTION OF THE DRAWINGS

FIG. 1 is a diagram showing the antimicrobial efficacy of chitosan-treated Kevlar®/Nomex® fabric vs. E. coli ATCC 25922, with and without prior surface treatment with base.

### DETAILED DESCRIPTION OF THE INVENTION

The present invention involves the preparation of antimicrobial ballistic and protective apparel, comprising aramid, polybenzazole, or high-performance polyethylene fiber that have a chitosan agent applied thereon.

The term “ballistic fabric” as used herein refers to a fabric that is suitable for use in garments, such as bulletproof vests, flack jackets and the like, which is designed to absorb the impact of a projectile and protect the wearer from harm. Ballistic fabrics have a wide variety of uses in addition to body armor and bulletproof vests. They are used for other types of protective clothing and equipment (e.g., bicycle and skateboarding helmets), marine and aircraft components, industrial belts, and recreational equipment such as sailing boats and clothes. Typical commercial examples of fiber used in making ballistic fabric include, but are not limited to, Kevlar® 29, Kevlar® 129, and Kevlar® Protera aramid, all available from E. I. du Pont de Nemours & Co., Inc. (Wilmington, Del.); Twaron® aramid fiber available from Teijin (Japan); Spectra® high performance polyethylene fiber (Honeywell International Inc., Morris Township, N.J.); Dyneema® high performance polyethylene fiber (DSM, Heerlen, the Netherlands). The term “ballistic fabric” includes not only fabric made from fibers but also composite materials used in, for example, concealable body armor and hard armor such as plates and helmets.

Concealable body armor is typically constructed of multiple layers of ballistic fabric. The layers are assembled into the “ballistic panel,” which in turn is inserted into a “carrier,” which is constructed of conventional garment fabrics such as nylon or cotton.

The term “protective apparel” as used herein refers to a garment or other article designed to protect the wearer from potential or actual harm and/or injury, but not specifically designed to absorb the impact of a projectile. One example is a firefighter turnout suit and hood that protects the user from high convective and radiant heat while firefighting. Other examples of protective apparel suitable for the present invention include, but are not limited to, knee pads, leg guards, face masks, gloves, breast plates, throat guards, protective cups, athletic supporters, shoulder pads, knee pads, elbow pads, wrist pads, radiation suits, self-contained breathing apparatus (for example, straps thereon), respirators (for example, webbing on face pieces), gas masks, and biological and chemical warfare suits and helmets. The present invention is especially suitable for inhibiting odor and/or fungal growth in the padding and/or lining materials of such protective garments.

The term “aramid” as used herein refers to an aromatic polyamide, wherein at least 85% of the amide (—CONH—) linkages are attached directly to two aromatic rings. Other flame retardant fibers (up to 40%) are blended with aramids to impact fabric thermal performance and comfort. Copolymers can be used having as much as 10 percent of other diamine substituted for the diamine of the aramid or as much as 10 percent of other diacid chloride substituted for the diacid chloride of the aramid. P-aramids are the primary polymers in fibers and fabrics of this invention, and poly(p-phenylene terephthalamide)(PPD-T) is the preferred p-aramid. M-aramids also find use in the present invention and poly (m-phenylene isophthalamide)(MPD-I) is the preferred m-aramid. M-aramid and p-aramid fibers and yarns particularly suitable for use in the present invention are those sold under the trademarks Kevlar® and Nomex® by E. I. du Pont de Nemours & Co., Inc. (Wilmington, Del.), Teijinconex®, Twaron®, and Technora® by Teijin (Japan) and equivalent products offered by others.

The term “polybenzazole” (PBZ) as used herein refers to an aromatic polymer comprising nitrogen-containing heterocyclic monomers. The structures, syntheses, and properties of polybenzazoles are described at length in numerous references, for example, in Materials Research Society Proceedings (1989), 134 (Mater. Sci. Eng. Rigid-Rod Polymn.), W. W. Adams, R. K. Eby, and D. E. McLemore (eds.) and U.S. Pat. Nos. 4,533,693 and 4,606,875. Preferred polybenzazoles for use in the present invention are poly(1,3-phenylenebisbenzimidazole) (PBI), poly(p-phenylene-2,6-benzobisoxazole) (PBO), poly(p-phenylenebisthiazole) (PBT), and poly(pyridobisimidazole) (PIPD, M5 fiber). PIPD is described in PCT Patent Application WO 94/25506 and D. J. Sikkema, Polymer, 39 (24), 5981-5986 (1998).

The term “high-performance polyethylene fiber” (HPPE) as used herein refers to high strength, oriented fiber produced from ultrahigh molecular weight polyethylene by a gel spinning process (see, e.g., U.S. Pat. Nos. 4,137,394 and 4,403,012 and Encyclopedia of Chemical Technology, 4th ed., J. I. Kroschwitz (exec. ed.), 13, 148ff (1995)). HPPE fibers are commercially available, for example, under the trademarks Spectra® (Honeywell International, Inc., Morris Township, N.J.) and Dyneema® (DSM, Heerlen, the Netherlands).

The textile definitions below are taken from the Complete Textile Glossary, Celanese Acetate LLC (2001).

The term “fiber” as used herein refers to a unit of matter, characterized by a length at least 100 times its diameter of width, which is capable of being spun into a yarn or made into a fabric by various methods such as weaving, knitting, braiding, felting and twisting.

The term “filament” refers to a fiber of an indefinite or extreme length.

The term “staple” or “staple fiber” refers to natural fibers or cut lengths from filaments. Manufactured fibers are cut to a definite length, from 8 inches down to about 1 inch, so that they can be processed on yarn spinning systems. The term “staple (fiber)” is used in the textile industry to distinguish natural or cut length manufactured fibers from filament.

The term “yarn” refers to a continuous strand of textile fibers, filaments, or material in a form suitable for knitting, weaving, or otherwise intertwining to form a textile fabric. For example, yarn can consist of staple fibers bound together by twist (“staple yarn” or “spun yarn”); many continuous filaments with or without twist (“multifilament yarn”); or a single filament with or without twist (“monofilament yarn”).

The articles of the present invention can be also be made using blends containing these fibers, such blends being made by methods commonly known in the art.

The present invention is particularly suited for use in fire fighter turnout gear. Such gear usually includes garments such as a coat and pants and any other articles of clothing needed to provide protection from heat and flame for the wearer. Generally, such garments are made of a series of individual components. Typically, such a garment has an outer shell usually made of abrasion resistant material, a moisture barrier made from water-resistant material, and a thermal barrier. PBI and both m- and p-aramids are commonly used in turnout coat outer shells.

Generally, the m-aramid fabric is used as a facing on the thermal barrier to increase durability. It is anticipated that the facing on this thermal barrier will be a preferred use of the fabric of the present invention. The fabric of the present invention can be used alone or in combinations with other fabrics in other types of protective garments. For example, the fabric may be used alone in a protective coat or coverall or as a lining for such garment.

The aramid fabrics useful in the present invention may be m-aramid, p-aramid, or mixtures thereof. For use as the inner lining of fire fighter turnout gear, it is especially preferred that the fabric of the present invention be a m-aramid fabric made, for example, from spun yarn or low friction filament. The fabric of the present invention may be woven or knitted. Although a plain weave or twill is preferred for most uses of this fabric, any weave pattern for the fabric or method of weaving or knitting the fabric may be used in making the fabric of the present invention.

In a fabric having more than 75% of the weight of the fabric as aramid yarns, the fabric has the character, at least in absorbing dye, fabric treatments and finishes, of an aramid fabric. Protective apparel uses may require some mix of multifilament or staple aramid yarns with other yarns, or may require that the protective fabric be 100% by weight aramid filament or multifilament yarns. For fabrics containing less than 100% to about 75% m-aramid fiber, the remaining fibers are selected for the required protective properties. Such yarns may be other yarns of high temperature stability such as p-aramid, amorphous m-aramid, (only available in fabrics) wool, FR rayon, and polybenzimidazole yarns. Such products tend to be intimate fiber blends.

The articles of the present invention are treated with chitosan to render them antimicrobial, thereby inhibiting the growth of fungus, bacteria, and the development of odor. Chitosan is the commonly used name for poly-[1-4]-β-D-glucosamine. Chitosan is chemically derived from chitin, which is a poly-[1-4]-β-N-acetyl-D-glucosamine that, in turn, is derived from the cell walls of fungi, the shells of insects and, especially, crustaceans. Articles comprising HPPE fiber can be treated with chitosan solution as in U.S. patent application Ser. No. 10/288,762, which is hereby incorporated by reference.

An article comprising aramid or polybenzazole fiber to be treated with chitosan first is soaked in water for about 5 to 30 minutes, preferably about 10 minutes. The temperature of the water is not critical; room temperature is preferred. The excess water is removed by suction or squeezing.

The article is then optionally soaked in the basic solution for about 5 to 30 minutes, preferably about 10 minutes, at room temperature to about 90 deg. C. The temperature of the water is not critical; room temperature is preferred. The basic solution comprises a base selected from the group consisting of soluble Group I hydroxides, soluble Group II hydroxides, soluble Group III hydroxides, ammonium hydroxide, and alkyl-substituted ammonium hydroxides; and the base is dissolved in water or a mixture of water with one or more water-soluble organic solvents selected from the group consisting of methanol, ethanol, propanol, ethylene glycol, propylene glycol, acetonitrile, dimethylformamide, and dimethylacetamide.

The article is then removed from the solution and excess basic solution is removed by suction under vacuum or squeezing. The article is then subjected to chitosan treatment.

The treatment comprises soaking or wetting the article with a solution containing a chitosan agent. The term “chitosan agent” as used herein means all chitosan-based moieties, including chitosan, chitosan salt, and chitosan derivatives. Preferably, the molecular weight of the chitosan agent is at least 50,000. It is also preferred that the degree of deacetylation of the chitosan is at least 85%.

The solution comprising the chitosan agent may be aqueous. However, since chitosan by itself is not soluble in water, the chitosan may be solubilized in a solution. Solubility is obtained by adding the chitosan to a dilute solution of a water-soluble, organic acid selected from the group consisting of mono-, di- and polycarboxylic acids. This allows the chitosan to react with the acid to form a water-soluble salt, herein referred to as “chitosan salt.” Alternatively, “chitosan derivatives,” including N- and O-carboxyalkyl chitosan, that are water-soluble, can be used directly in water instead of chitosan salt.

Typically, the chitosan solution is an aqueous acetic acid solution, for example, an aqueous solution containing 2% chitosan and 0.75% acetic acid or 2% chitosan and 1.5% aqueous acetic acid. The time of treatment is typically 5 to 30 minutes. The temperature of the treatment is not critical, room temperature being preferred. After treatment with chitosan solution, excess solution may be allowed to drip out, or may be removed by wringing or spinning. The treated article is then dried via oven drying or a combination of ambient air-drying and oven drying. The resulting coating exhibits satisfactory durability when laundered.

Articles prepared by the above methods exhibit antimicrobial and anti-odor properties. The term “antimicrobial” as used herein, means both bactericidal and fungicidal as is commonly known in the art. By “antimicrobial growth is reduced” or “reduction of bacterial (or fungal) growth” is meant that a 99.9% kill of the bacteria (or fungus) in 24 hours has been met as measured by the Shake Flask Test described below and as is commonly used to measure antimicrobial functionality which indicates a minimum requirement of a 3-log reduction in bacterial growth.

In addition, the fibers and fabrics processed herein exhibit favorable physical properties with respect to tenacity, elongation and hand-feel.

Said antimicrobial properties may, optionally, be further enhanced by treatment with soluble metal salts, for example, soluble silver salts, soluble copper salts and soluble zinc salts. The preferred metal salts of the invention are aqueous solutions of zinc sulfate, copper sulfate or silver nitrate. The metal salts are typically applied by dipping or padding a dilute (0.1 to 5%) solution of salt in water. The degree of enhancement depends on the particular metal salt used, its concentration, the time and temperature of exposure, and the specific chitosan treatment, that is, the type of chitosan agent, its concentration, the temperature, and the time of exposure.

In a preferred embodiment, the process of the present invention further involves heating the chitosan-coated article comprising polybenzazole or aramid fiber to a temperature in the range 30° C. to 200° C. under a nitrogen or ambient atmosphere for from 30 minutes to 18 hours. It is preferred that the chitosan coating remain substantially uncrosslinked.

The aramid or polybenzazole article to be treated according to the present invention, particularly in the form of fiber, yarn, or fabric can also be rendered antimicrobial by means of a continuous process, comprising the sequential steps of:
(a) providing a feed station on which is disposed the article and a take-up station capable of receiving the article;
(b) drawing the article from the feed station through a first treatment station wherein said article is contacted with water;
(c) optionally, drawing the article from the first treatment station through a second treatment station wherein said article is exposed to a basic solution;
(d) drawing the step (b)- or step (c)-treated article through a third treatment station wherein the article is exposed to a solution comprising a chitosan agent selected from the group consisting of chitosan, chitosan salts and chitosan derivatives;
(e) drawing the step (c)- or step (d)-treated article through a drying station wherein the article is exposed to a temperature of 120° C.;
(f) optionally, heating the step (e)-treated article at 200° C. after it exits the drying station; and
(g) causing the step (e)- or step (f)-treated article to be received on and accumulate on the take-up station.

The feed station, treatment stations, heaters, and take-up components may be any convenient means known in the art for continuous treatment of fibers and yarns (see, for example, Ullmann's Encyclopedia of Industrial Chemistry, Fifth Edition, Wolfgang Gerhartz, Executive Editor, Volume A10, VCH Verlagsgesellschaftg, Weinheim, Federal Republic of Germany (1987), “Fibers, 3. General Production Technology,” H. Lucker, W. Kagi, U. Kemp, and W. Stibal, pp. 511-566). The continuous process is particularly appropriate for use on a commercial scale.

The chitosan-treated articles of the present invention are in the form of fiber, yarn, woven and nonwoven fabric, film; and articles and constructs prepared therefrom.

The articles of the present invention find use in body armor; fire fighter gear, such as jackets, hoods, overalls, and gloves, especially thermal liners for such articles; industrial garments for electrical, chemical, and petrochemical protection; rainwear; limited use protective garments; cut- and heat-resistant gloves, hoods, and helmet liners; radiation suits, self-contained breathing apparatus (for example, straps thereon), respirators (for example, webbing on face pieces), gas masks, and biological and chemical warfare suits and helmets; fire blocking for mattresses, upholstery, and transportation seating; auto race-car suits and liners for same; military garments for tank crews and pilots; insulation for boots, gloves, and cold weather gear; jeans, cut-and ballistic-resistant outdoor clothing, chainsaw chaps; and athletic protective gear such as knee pads, arm pads, rib pads, leg guards, face masks, chin pads, gloves, chest protectors, breast plates, throat guards, protective cups, athletic supporters, shoulder pads, elbow pads, and wrist pads. The present invention is especially suitable for inhibiting odor and/or fungal growth in the padding and/or fabric lining of protective garments.

### EXAMPLES

### Example 1

Material: A sample of Kevlar®/Nomex® aramid batt thermal liner of the type used in the protective gear of a typical firefighter uniform was obtained from E. I. du Pont de Nemours & Co., Inc. (Wilmington, Del.). The batt consisted of 75% Kevlar® and 25% Nomex® fibers. The batt was stitched to a face cloth consisting of Nomex®. Pieces (4″×4″) were cut from the large piece and used in the following experiments.

Chitosan Deposition of Sodium Hydroxide treated Kevlar®/Nomex®: Two pieces of the above fabric (A=2.43 g, B=2.51 g) were soaked in water for 30 min. The excess water was drained by suction, and the fabric pieces were then soaked in 10% aqueous sodium hydroxide solution for 30 min. The pieces were then taken out and excess sodium hydroxide solution was removed by suction under vacuum. The pieces were then soaked in 2% chitosan (ChitoClear® TM656, obtained from Primex, Norway, molecular weight about 70,000, degree of deacetylation over 90%) solution in 0.75% aqueous acetic acid for 30 min. The pieces were then taken out and the excess chitosan solution was removed by suction under vacuum. The pieces were then dried in air for 24 h, followed by heating to 110° C. for 1 h. Weights of the fabrics at the end were A=2.72 g, B=2.73 g.

Chitosan Deposition on Kevlar®/Nomex® fabric: Two pieces of the above fabric (C=2.62 g, D=2.59 g) were soaked in water (30 min.). The excess water was drained by suction, and the pieces were then soaked in 2% chitosan (ChitoClear® TM656, obtained from Primex, Norway, molecular weight about 70,000, degree of deacetylation over 90%) solution in 0.75% aqueous acetic acid for 30 min. The pieces were then taken out and the excess chitosan solution was removed by suction under vacuum. The pieces were then dried in air for 24 h, followed by heating to 110° C. for 1 h. Weights of the fabrics at the end were C=2.79 g, D=2.72 g.

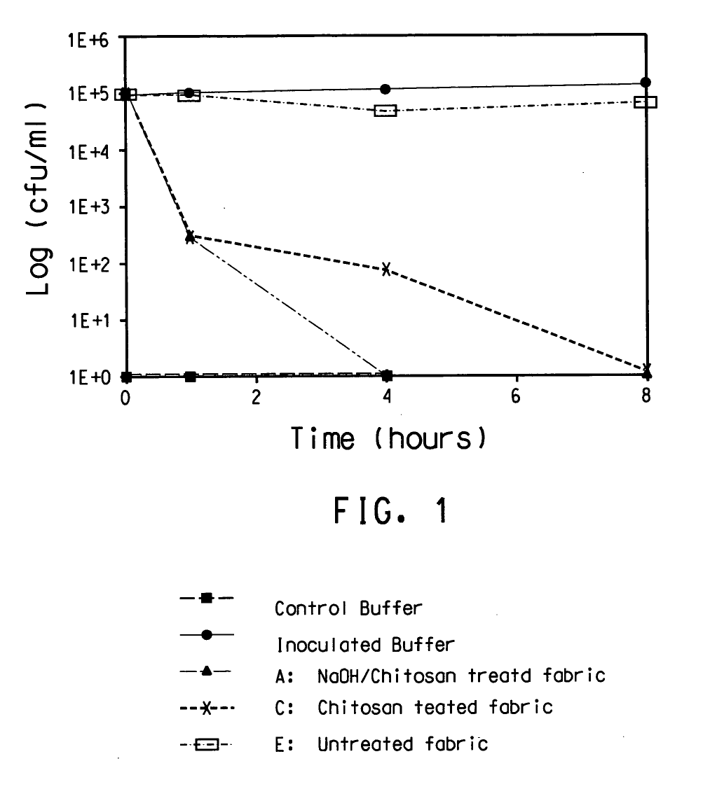

The samples A and C were tested along with an untreated fabric (sample E) for antimicrobial efficacy against E. Coli ATCC 25922 and the results are shown in FIG. 1. Based on the results it can be concluded that the prior treatment of the fabric with sodium hydroxide increases the antimicrobial efficacy. The sodium hydroxide primed chitosan padded fabric (filled triangles) reduces the bacterial colonies more rapidly as compared to the nonprimed fabric (X's).

### Example 2

Ten 4 inch by 4 inch (10 cm by 10 cm) pieces of the Kevlar®/Nomex® aramid batt thermal liner material described in Example 1 were cut, placed in a container with 1500 ml water and rolled on a tumbler for 30 minutes. The pieces were removed and excess water was drained by suction while squeezing. The pieces were then placed in a second container with 1500 ml 10% NaOH aqueous solution and rolled on a tumbler for 30 minutes. The pieces were removed and excess liquid was drained by suction while squeezing. The pieces were washed once with water to remove excess NaOH. The pieces were then put in a third container with 1500 ml of a 2% chitosan (ChitoClear® TM656, obtained from Primex, Norway, molecular weight about 70,000, degree of deacetylation over 90%) solution in 0.75% aqueous acetic acid and rolled on a tumbler for 30 minutes. The pieces were removed and excess liquid was drained by suction while squeezing. The pieces were hung in ambient air overnight to dry. The pieces were then put into a preheated 110° C. oven for 1 hour, removed, and allowed to cool. They were put into a container with 3 liters water, rolled on a tumbler for 4 hours, removed, and hung in ambient air to dry overnight. The indicating azo dye Orange II 0.5 g/l in 0.7% aqueous acetic acid) was used to confirm the presence of chitosan at the surface of these treated fabric pieces and its absence at the surface of untreated fabric.

Treated and untreated samples were laundered according to AATCC Test Method 61, Test C, subjected to 5, 10, or 30 wash cycles. Additional treated and untreated samples were subjected to 5, 10, or 30 wash cycles with Tide® laundry detergent (Proctor & Gamble).

Fabric samples were soaked in Orange II dye, washed with water, and air-dried. The intense orange coloration indicated the presence of chitosan on the treated fabric samples. Fabric that was untreated did not exhibit such coloration when similarly treated with Orange II dye. After 5, 10, and 30 wash cycles, all the chitosan-treated fabrics were still orange, indicating a considerable amount of chitosan still coated the surface of the polyester fabric. The intensity of the orange hue diminished significantly after the 30 wash cycles with AATCC. There was only a small fading of the orange hue at the surface of the samples subjected to 30 wash cycles with Tide® laundry detergent.

1. A method for rendering an article comprising aramid or polybenzazole antimicrobial, comprising the sequential steps:
(a) providing the article comprising aramid or polybenzazole;
(b) contacting said article with water;
(c) optionally, contacting the article with a basic solution;
(d) contacting the article produced in step (b) or step (c) with a solution comprising a chitosan agent selected from the group consisting of chitosan, chitosan salts and chitosan derivatives;
(e) drying the article; and
(f) optionally, heating the dried article at a temperature in the range of 30° C. to 200° C. for 30 minutes to 18 hours.

2. A continuous method for rendering an article comprising aramid or polybenzazole antimicrobial, comprising the sequential steps:
(a) providing a feed station on which is disposed the article and a take-up station capable of receiving the article;
(b) drawing the article from the feed station through a first treatment station wherein said article is wet with water;
(c) optionally, drawing the article from the first treatment station through a second treatment station wherein said article is exposed to a basic solution;
(d) drawing the step (b)- or step (c)-treated article through a third treatment station wherein the article is exposed to a solution comprising a chitosan agent selected from the group consisting of chitosan, chitosan salts and chitosan derivatives;
(e) drawing the step (c)- or step (d)-treated article through a drying station;
(f) optionally, heating the step (e)-treated article at a temperature of from 30° C. to 200° C. after it exits the drying station; and
(g) causing the step (e)- or step (f)-treated article to be received on and accumulate on the take-up station.

3. The method of claim 1 or claim 2 wherein the basic solution comprises a base selected from the group consisting of soluble Group I hydroxides, soluble Group II hydroxides, soluble Group III hydroxides, ammonium hydroxide, and alkyl-substituted ammonium hydroxides; and the base is dissolved in water or a mixture of water with one or more water-soluble organic solvents selected from the group consisting of methanol, ethanol, propanol, ethylene glycol, propylene glycol, acetonitrile, dimethylformamide, and dimethylacetamide.

4. The method of claim 1 or claim 2 wherein the solution comprising a chitosan agent is a solution of chitosan in a dilute, water-soluble, organic acid selected from the group consisting of mono-, di- and polycarboxylic acids.

5. The method of claim 1 or claim 2 wherein the solution comprising a chitosan agent is chitosan in dilute aqueous acetic acid.

6. The method of claim 5 wherein said solution comprises 0.25% to 5.0% by volume of the dilute aqueous acetic acid and 0.25% to 8.0% of chitosan by weight of the solution.

7. The method of claim 1 further comprising contacting the article produced in step (d), (e), or (f) with a solution comprising a metal salt; a solution comprising a carboxyl-containing polymer; an additional solution comprising a chitosan agent; or combinations thereof, wherein the surface of the article produced comprises chitosan, a metal salt, or combinations thereof.

8. The method of claim 2 further comprising drawing the step (d)-, (e)-, (f)-, or (g)-treated article through a subsequent station containing a solution comprising a metal salt; a solution comprising a carboxyl-containing polymer; an additional solution comprising a chitosan agent; or combinations thereof, wherein the surface of the article produced comprises chitosan, a metal salt or combinations thereof.

9. The method of claim 7 or claim 8 wherein the metal salt is selected from the group consisting of soluble silver salts, soluble copper salts, and soluble zinc salts.

10. The method of claim 7 or claim 8 wherein the metal salt is selected from the group consisting of silver nitrate, copper sulfate, and zinc sulfate.

11. The method of claim 7 or claim 8 wherein the carboxyl-containing polymer is polyacrylic acid or sodium carboxymethylcellulose.

12. The method of claim 1 or claim 2 wherein the article is in the form of a filament, fiber, yarn, fabric or film.

13. The method of claim 1 or claim 2 wherein the aramid is selected from the group consisting of poly(p-phenylene terephthalamide), poly(m-phenylene terephthalamide), and copolymers and blends thereof.

14. An article comprising aramid or polybenzazole, wherein the surface of said article has been treated with a chitosan agent.

15. An article comprising high-performance polyethylene, wherein the surface of said article has been treated with a chitosan agent.

16. The article of claim 14 prepared according to the method of claim 1 or claim 2.

17. A ballistic fabric comprising aramid, polybenzazole, or high-performance polyethylene fiber that has been treated with a chitosan agent.

18. The article of claim 14, which is a protective garment.

19. The article of claim 15, which is a protective garment.

20. A protective garment having a lining or a padding, wherein the lining or the padding comprises aramid, polybenzazole, or high-performance polyethylene fiber that has been treated with a chitosan agent.

21. The protective garment of claim 18 or claim 19 which is a coat; a jacket; an overall; a body armor; a limited-use protective garment; a glove; a boot; a military garment; an auto race-car suit; jeans; chain-saw chaps; cut-resistant outdoor clothing; or ballistic outdoor clothing.

22. The protective garment of claim 18, which is a fire suit; a radiation suit; an industrial garment for protection from electricity, chemicals or petrochemicals; rainwear; or a protective garment for chemical and biological warfare.

23. A helmet, hood, self-contained breathing apparatus, respirator, or gas mask comprising aramid, polybenzazole, or high-performance polyethylene that has been treated with a chitosan agent.

24. A helmet, hood, self-contained breathing apparatus, respirator, or gas mask having a lining, straps, or webbing, wherein the lining, straps, or webbing comprises aramid, polybenzazole, or high-performance polyethylene that has been treated with a chitosan agent.

25. The article of claim 14 or claim 15, which is an athletic protective article.

26. The athletic protective article of claim 25 which is a knee pad, elbow pad, rib pad, arm pad, shoulder pad, wrist pad, leg guard, shin guard, glove, face mask, chin pad, breast plate, chest protector, throat guard, protective cup, or athletic supporter.

27. An athletic protective article having a lining or a padding, wherein the lining or padding comprises aramid, polybenzazole, or high-performance polyethylene fiber that has been treated with a chitosan agent.

28. Fire blocking material for mattresses, upholstery, or transportation seating comprising the article of claim 14.

29. A lining or padding material for mattresses, upholstery, or transportation seating wherein said lining or padding comprises aramid that has been treated with a chitosan agent.

30. The method of claim 1 or claim 2 wherein the molecular weight of the chitosan agent is at least 50,000.

31. The method of claim 1 or claim 2 wherein the degree of deacetylation of the chitosan agent is at least 85%.

32. The article of claim 14 or claim 15 wherein the molecular weight of the chitosan agent is at least 50,000.

33. The article of claim 14 or claim 15 wherein the degree of deacetylation of the chitosan agent is at least 85%.

34. The article of claim 14 or claim 15 wherein the chitosan agent is substantially uncrosslinked.
